(19) United States
(12) Patent Application Publication
(10) Pub. No.: US 2008/0131869 A1
(43) Pub. Date: Jun. 5, 2008

(54) Method For Detecting An Analyte

(75) Inventors: Filip Frederix, Hasselt (BE); Gustaaf Borghs, Leuven (BE); Jean-Michel Friedt, Neuilly sur Seine (FR)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: INTERUNIVERSITAIR MICROELEKTRONICA CENTRUM (IMEC), Leuven (BE)

(21) Appl. No.: 11/769,237

(22) Filed: Jun. 27, 2007

Related U.S. Application Data
(63) Continuation of application No. 10/322,901, filed on Dec. 18, 2002, now abandoned.
(60) Provisional application No. 60/345,169, filed on Dec. 21, 2001.

(51) Int. Cl. C12Q 1/70; G01N 21/62; G01N 33/53; C12Q 1/68

(52) U.S. Cl. 435/5; 436/171; 435/7.32; 435/7.1; 435/6

(57) ABSTRACT

The present invention discloses an improved method for detecting an analyte. The present invention may be used for sensing devices which have a higher sensitivity and which can be used to detect very low concentration of analyte. In one embodiment, the method comprises the steps of providing a substrate, said substrate comprising a conductive region and a recognition layer, said conductive region having at least a first surface and a second surface, wherein said first surface is operatively associated with said recognition layer; subjecting said substrate to said analyte such that an interaction occurs between said analyte and said recognition layer; directing radiation through said substrate such that said radiation incidents on said conductive region and said recognition layer; and measuring the intensity of said radiation absorbed or transmitted by said substrate as a function of the wavelength in order to determine the presence of an analyte.

### RELATED APPLICATION

This application is a continuation of U.S. patent application Ser. No. 10/322,901, filed on Dec. 18, 2002, which claims priority to U.S. Provisional Patent Application Ser. No. 60/345,169, filed on Dec. 21, 2001, the disclosures of which are incorporated herein by reference.

### FIELD OF THE INVENTION

This invention relates generally to assaying. More specifically, it relates to a method for assaying an analyte.

### BACKGROUND OF THE INVENTION

Different types of biosensors are known with their specific advantages and disadvantages. Electrochemical biosensors, Surface Acoustic Wave sensors and Surface Plasmon Resonance biosensors are examples of biosensors which do not require labelling techniques.

U.S. Pat. No. 5,641,640 discloses a method for assaying an analyte in a fluid sample using surface plasmon resonance. The presence of an analyte is determined by the change in refractive index when the analyte interacts with a refractive index enhancing species. Surface plasmon resonance measurements have some major disadvantages such as most systems are rather expensive. The system requires a quartz prisma and requires a radiation source that is capable to generate polarized light. Also, the SPR response depends on the volume and refractive index of the bound analyte. For very small molecules, this results in very small changes of refractive index. Further, the receptors should be immobilized on the surface.

U.S. Pat. No. 6,330,464 discloses an optical based sensing device for detecting the presence of an amount of analyte using boh indicator and reference channels. The sensor has a sensor body with a source of radiation therein. Radiation emitted by the source interacts with molecules, resulting in a change of at least one optical characteristic of those molecules.

Therefore, there exists a need for an improved method of detecting an analyte.

### SUMMARY OF THE INVENTION

In a first aspect of this invention, a method for analysing and determining an analyte within a sample is disclosed. In one embodiment, the method includes providing a substrate, said substrate comprising a conductive region and a recognition layer, said conductive region having at least a first surface and a second surface, wherein said first surface is operatively associated with said recognition layer; subjecting said substrate to said analyte, such that an interaction occurs between said analyte and said recognition layer; directing radiation through said conductive region and said recognition layer; and measuring the intensity of said radiation absorbed or transmitted by said substrate as a function of the wavelength in order to determine the presence of an analyte. Said method can be used for affinity immunosensing and biosensing in general by optically monitoring of the recognition layer deposition, antibody immunization and the recognition of the antigen.

In an embodiment of the first aspect of this invention, a method as recited in the first aspect of this invention is recited wherein said step of subjecting said substrate to said analyte is performed such that an interaction occurs between said analyte and said recognition layer

In an embodiment of the first aspect of this invention, a method as recited in the first aspect of this invention is disclosed wherein said conductive region consists essentially of at least one particle. Said particle is preferably smaller than the wavelength of the impinging radiation. The interaction between the analyte and the conductive regions affects the dielectric constant of the conductive region and the recognition layer, resulting in a change in the absorption or transmittance spectrum. Moreover, the interaction results in a change in the resonance frequency of the particle plasmon, since this is mainly determined by the dielectric function of the conductive region and the surrounding medium such as the recognition layer and the particle shape.

In a further embodiment, a method as recited in any of the previous embodiments of the first aspect of this invention is disclosed wherein said diameter of the particle is below 300 nm, below 200 nm, below 100 nm or below 50 nm.

In a further embodiment, a method as recited in any of the previous embodiments of the first aspect of this invention is disclosed wherein said interaction between said analyte and said recognition layers results in a change of the dielectric constant of said recognition layer.

In a further embodiment, a method as recited in any of the previous embodiments of the first aspect of this invention is disclosed wherein said substrate further comprises a support layer, and wherein said second surface is operatively associated with said support layer. Said said support layer is transparent or semi-transparant.

In a further embodiment, a method as recited in any of the previous embodiments of the first aspect of this invention is disclosed wherein said conductive region comprises a metal. Said metal can be a metal inducing a plasmon effect. Said metal can be selected from the group consisting gold, silver and copper.

In a further embodiment, a method as recited in any of the previous embodiments of the first aspect of this invention is disclosed wherein said recognition layer comprises a self-assembling monolayer.

In a further embodiment, a method as recited in any of the previous embodiments of the first aspect of this invention is disclosed wherein said substrate has multiple conductive regions and wherein said conductive regions are ordered in an array. Said substrate can be a microtitre plate. Said substrate can be used for high-throughput screening.

In a further embodiment, a method as recited in any of the previous embodiments of the first aspect of this invention is disclosed further comprising the steps of providing a second substrate, said second substrate comprising a conductive region having at least a first surface and a second surface, and a recognition layer, wherein said first surface is operatively associated with said recognition layer; subjecting said second substate substrate to a reference sample; directing radiation through said conductive region and recognition layer of said second substrate; measuring the intensity of said radiation absorbed or transmitted by said second substrate as a function of the wavelength; and comparing the intensity of said radiation absorbed or transmitted by said second substrate with the intensity of said radiation absorbed or transmitted by said first substrate in order to determine the presence of an analyte.

Therefore, it is an object of this invention to provide a method for assaying an analyte, which requires no labelling and which is very sensitive. It is a further an object of this invention to provide a method which is user-friendly and has a low manufacturing cost price. It is also an object of the invention to use the method for assaying biomolecules.

### DETAILED DESCRIPTION OF THE DRAWINGS

The presently preferred embodiments of the present invention are described herein with reference to the drawings, in which:

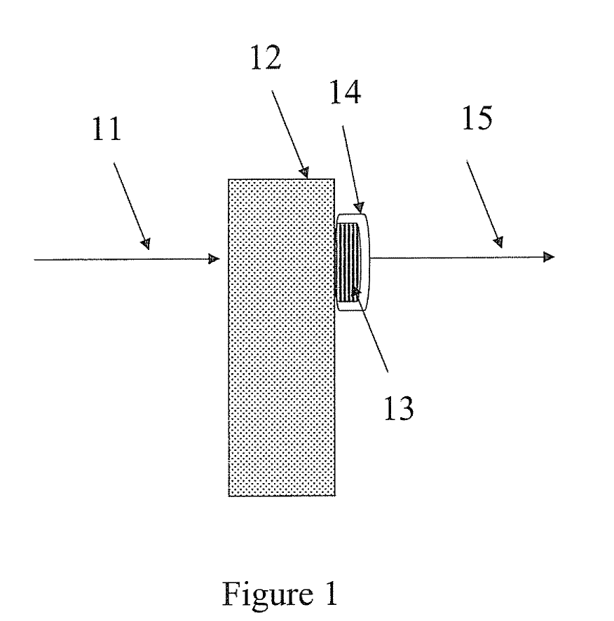

FIG. 1 is an experimental set-up as used in an embodiment of the method of the present invention;

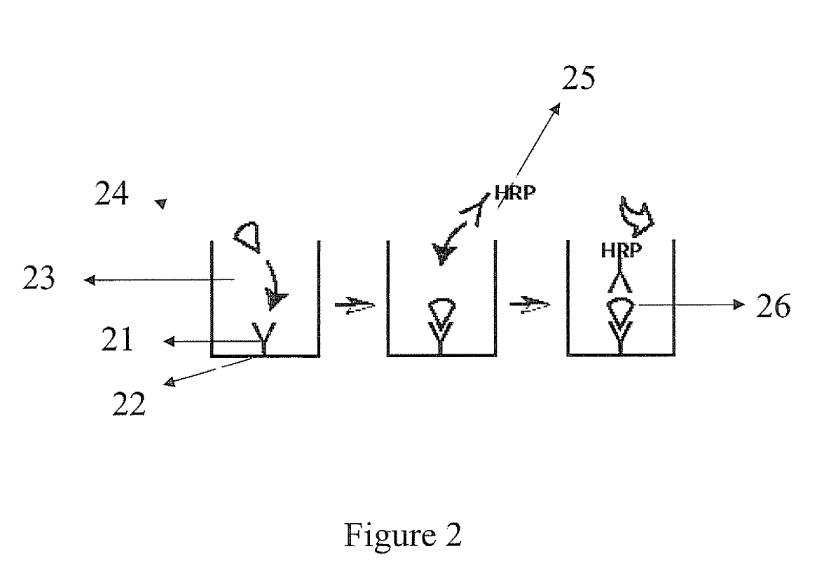

FIG. 2 is a schematic representation of the method as described in conventional ELISA experiments;

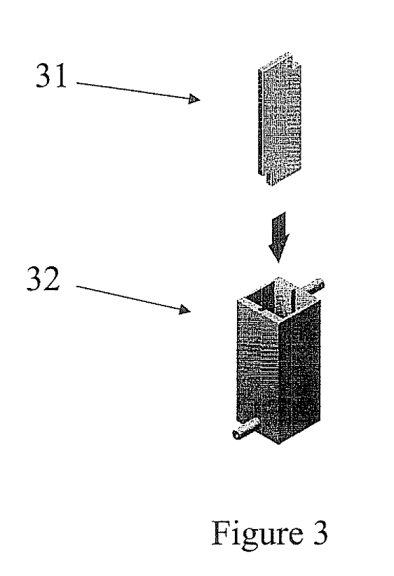

FIG. 3 is a schematic representation of slides and the quartz cells as described in the preferred embodiment of the present invention;

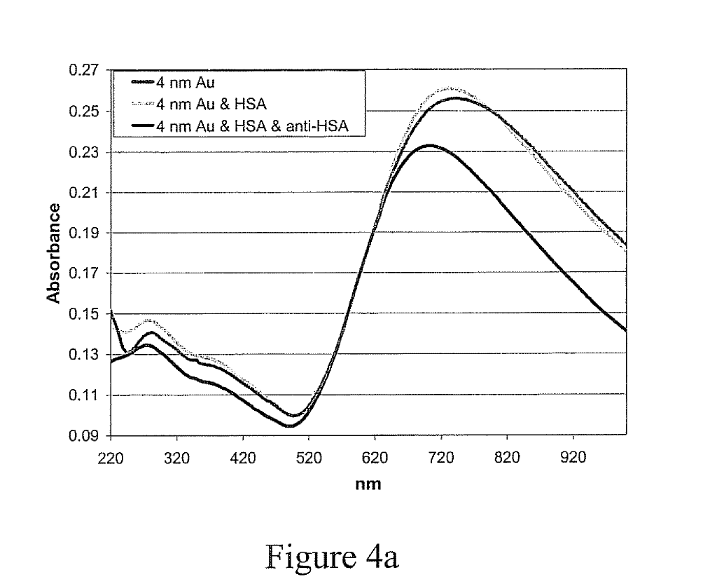

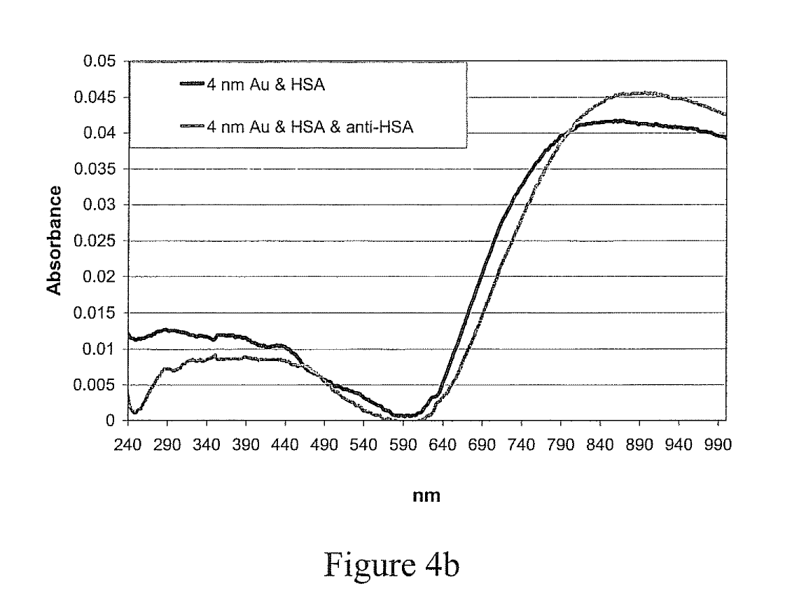

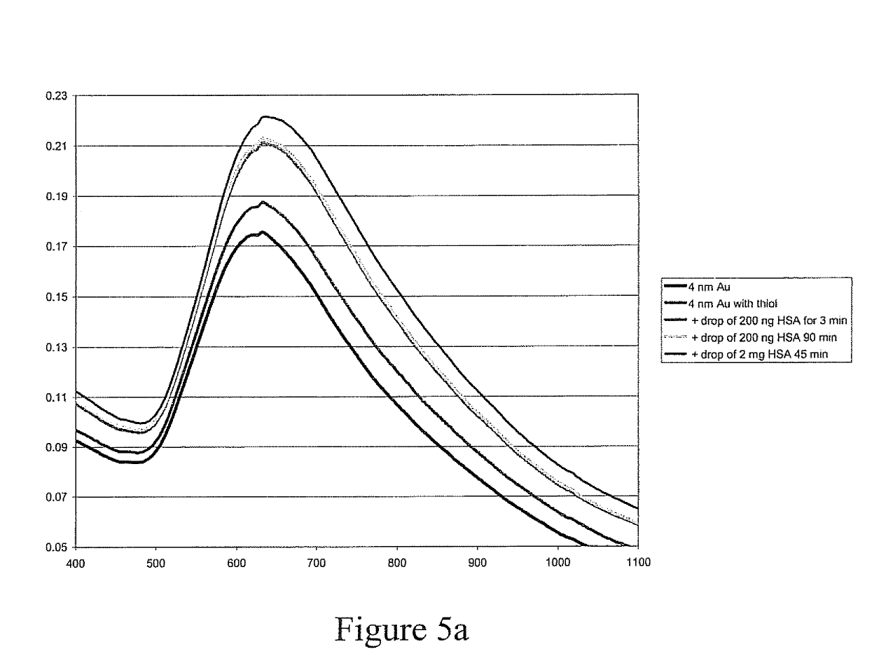

FIG. 4(a) is an absorbance spectra of Human Serum Albumin directly adsorbed on the thin gold film;

FIG. 4(b) is a difference spectra Human Serum Albumin directly adsorbed on the thin gold film and a thin gold on quartz;

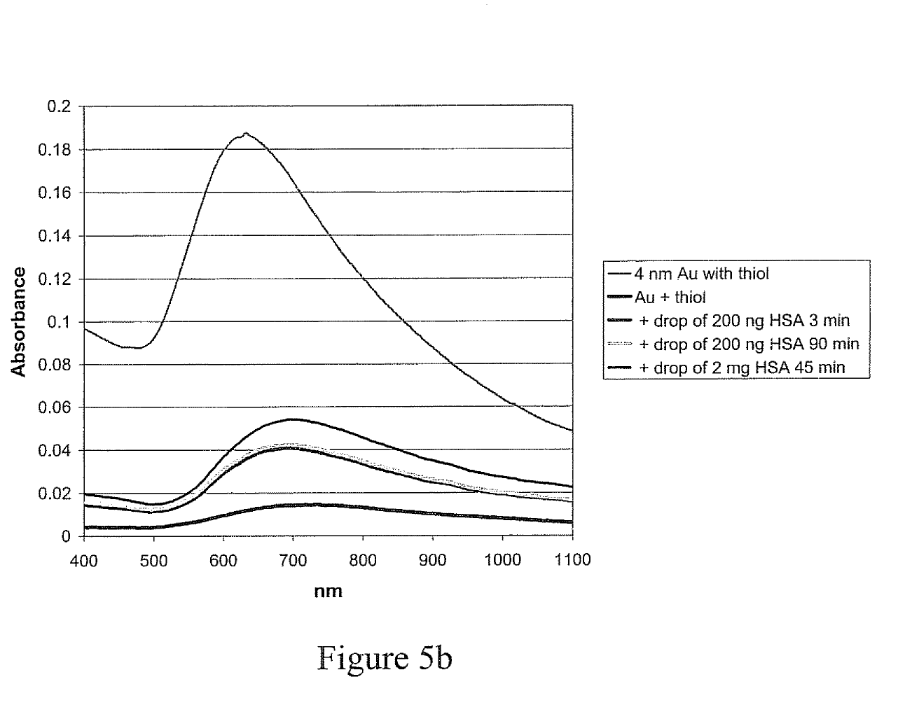

FIG. 5(a) is an absorbance spectra of self-Assembeld Monolayers of thiols on gold followed by adsorption of HSA at different concentrations and different times;

FIG. 5(b) is a difference spectra of the spectra of FIG. 5(a); and

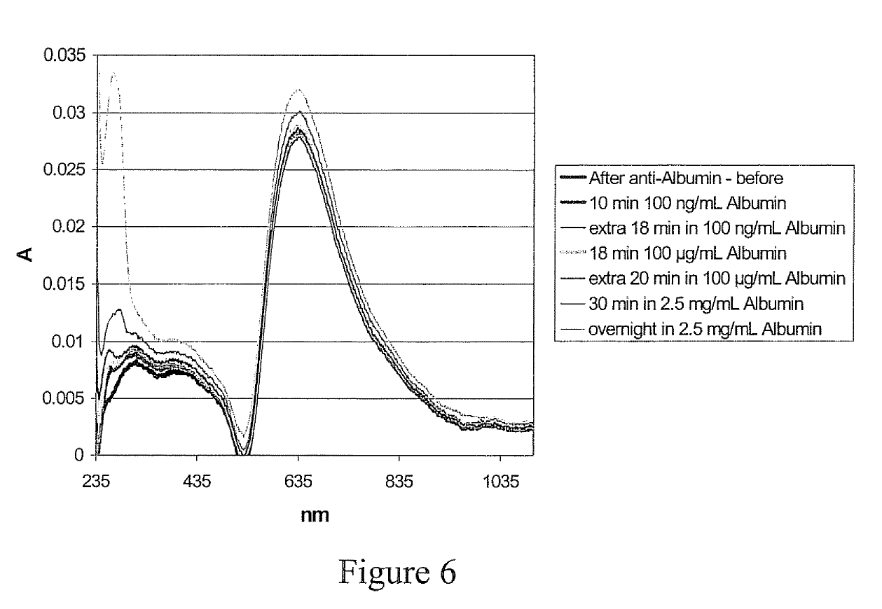

FIG. 6 is a difference spectra used in immunosensing applications

### DETAILED DESCRIPTION OF PREFERRED EMBODIMENTS

The present invention discloses an improved method for detecting an analyte. The present invention may be used, for example, in sensing devices which have a higher sensitivity and which can be used to detect very low concentrations of analyte.

In particular, this method can be used for affinity immunosensing and biosensing in general by optically monitoring of the linking layer deposition, recognition molecule immobilization and recognition of the analyte. This method is versatile, allowing applications in the liquid, gas phase, and allows quantitative in situ measurements.

The present invention discloses a method for assaying an analyte in a sample, wherein said sample is brought into contact with a substrate comprising a conductive region and a recognition layer. The presence of an analyte being determined by the resulting change in the spectrum (wavelength/frequency vs. intensity) of the light transmitted through the substrate or absorbed by the substrate in contact with sample.

For the purpose of this invention, it should be understood that the words “absorbed radiation (or absorbance)” and “transmitted radiation (or transmittance)” can replace each other. The relation between the absorbance (A) and the transmittance (T) is given by: A=−log T

In a first aspect of this invention, a method for analysing and determining an analyte within a sample is disclosed comprising the steps of providing a substrate, said substrate comprising a conductive region and a recognition layer, said conductive region having at least a first surface and a second surface, wherein said first surface is operatively associated with said recognition layer; subjecting said substrate to said analyte such that an interaction occurs between said analyte and said recognition layer; directing radiation through said substrate such that said radiation incidents on said conductive region and said recognition layer; and measuring the intensity of said radiation absorbed or transmitted by said substrate as a function of the wavelength in order to determine the presence of an analyte. Said substrate can further comprise a support layer, said second surface of said conductive region can be operatively associated with said support layer.

The present invention can be used for assaying for an analyte in a sample. Said analyte can be any kind of chemical molecule such as, but not limited hereto, a biomolecule, ions, and cells. Said biomolecule can be, for example, hormones, proteins such as antibodies, antigens, steroids, nucleic acids, drug metabolites or microorganismes. Said sample can also be a “blank” sample, such as a sample without analyte, or no sample.

Said source of radiation can be any radiation source such as a lamp, a light emitting diode (LED) or a laser In an embodiment, said radiation source should be able to provide a substantial amount of radiation in the wavelength range of the maximum absorbance (or minimum transmittance) wavelength of the substrate. Preferably, said radiation source provides radiation with a wavelength between 200 nm and 1500 nm. Preferably, said radiation source can generate radiation with a wavelength between 200 nm and 1000 nm. A LED, like a red LED or a blue LED can be used. Also other sources of radiation can be used. Said radiation source can be a radiation source with a focussed beam (such as e.g. laser) or can be a light source providing light with a broader spectrum. Preferably, said radiation source provides collimated radiation Said radiation can be monochromatic.

Between said source of radiation and said substrate, several components can be present, such as, but not limited hereto, lenses, slits, gratings. The radiation source can be part of a commercially available UV-VIS spectrometer (or absorptiometer or colorimeter).

Said substrate generally comprises a conductive region and a recognition layer. Said substrate can further comprise a support layer. Said support layer is for achieving at least mechanical support. Said support layer could be transparent or semi-transparent for the wavelength provided by the radiation source. Said support layer should have an optical transparency between 5% and 95%, preferably between 20% and 80%, preferably, at least 80%. Said substrate can be made of, but is not limited hereto, glass, quartz, polymeric material (such as polycarbonate, polysulphonate, polymethyl-methacrylate). Preferably, said support layer is made of glass or quartz. Said support layer can be flat. Said support can also be part of a glass or quartz tube, a polymeric tube or a micro-titre plate. The substrate can be integrated in a flow system. The substrate can also be a microtitre plate being part of a high-throughput screening system or ELISA tests.

Said conductive region comprises a first surface and a second surface. The first surface is operatively associated with said recognition layer. The second surface can be in contact with an external medium (such as e.g. air or a gas). In a preferred embodiment, the second surface is operatively associated with the support layer.

The conductive region comprises a conductive material. Preferably, said conductive material is a metal. Said metal can be, but is not limited hereto, gold, silver, copper. Any metal inducing a plasmon effect can be used of this invention. Other possible materials include conductive glass, conductive polymers or metallic nanoparticles.

Said conductive region can be a conductive layer or can be at least one particle. A conductive layer can have a thickness below 60 nm, below 50 nm, below 40 nm, below 30 nm below 20 nm or below 10 nm and preferably below 5 nm. Said layer can be continuous or can be discontinue such that islands of conductive material are formed. A continuous layer can be uneven or even.

More preferably, said conductive region consists essentially of particles, more particularly micro- or nanoparticles. The size of the particles is lower than the wavelength of the radiation that incidents on the particle. The diameter of the particles is lower than 500 nm, lower than 400 nm, preferably lower than 300 nm. Said thickness can also be lower than 200 nm, lower than 100 nm, lower than 80 nm, lower then 50 nm, lower than 40 nm, lower than 30 nm or lower than 20 nm. The size of the particles can be determined by the deposition process. The shape of the particles can be spherical, but other structural and spatial configurations are not excluded. For instance, the particles can be slivers, cubic, ellipsoids, tubes and the like. The particles can be hollow. The particle can consist essentially of conductive material. The particle can also consist of a polymeric material covered with conductive material.

Said conductive region can be adapted in such a way that it can be optically tuneable. Optically tuneable layer means that the region has been produced in such a way that it has a predetermined thickness or a predetermined particle size, which corresponds to a preset value of the wavelength where the intensity of the absorbed radiation is sufficient high. The desired thickness of the conductive region can be controlled by evaporation, sputtering, electroless plating or electroplating the conductive material.

In an embodiment, the second surface of the conductive region is operatively associated with a support layer. In an embodiment, the second surface of the conductive region can be deposited directly on the support layer. In another embodiment, at least one adhesion layer can be present between the support layer and the second surface of the conductive region. Said adhesion layer can improve the stability of the conductive region. Said adhesion layer can be, but is not limited hereto, a layer of self-assembling molecules such as, but not limited hereto, silane-based molecules or thiol-based molecules. Said adhesion layer can also comprise a layer of organic linker molecules molecules (e.g. glue, etc. . . . ). Said adhesion layer can, but does not have to have an effect on the absorption/transmittance characteristics of the first layer. Preferably, said adhesion layer is a non-metallic layer.

Said substrate can further comprise said recognition layer. Said recognition layer is operatively associated with the first surface of the conductive region. Said recognition layer is a layer comprising at least recognition molecules, also called receptive molecules. Said recognition molecules comprise one part of a specific binding pair and include anti-gen/antibody, enzyme/substrate, metal/chelator, bacteria/receptor, virus/receptor, hormone/receptor, oligonucleotide/RNA, DNA/RNA, RNA/RNA, oligonucleotide/DNA. The receptor molecules should be capable to specifically interact with the analyte. This interaction can result in a change of the dielectric constant of the conductive region and the recognition layer. The recognition layer can also be a layer of cells being deposited directly on the first layer or on an intermediate layer. For the purpose of this application, intermediate layer should be understood as a layer being formed on the first surface of the conductive region. In many cases, the recognition layer is designed such that non-specific adsorption is essentially avoided.

Said recognition molecules can be deposited directly on the first surface of the conductive layer. Said recognition layer can also comprise a self-assembled monolayer (SAM) on which the recognition molecules can be bound (covalent or physical adsorption). Said Self-Assembled Monolayer can comprise at least two functional group, a first group is be selected such that it is operatively associated with the first surface of the conductive region and a second group is selected such that it interacts with the analyte. An interaction between the recognition molecule and the analyte can, but does not necessary have to, result in a change of the absorption spectrum of the substrate.

When the substrate is subjected to the sample, the substrate is subjected to the radiation source such that the incident light impinges on the substrate, in particular on the conductive region and the recognition layer. The transmittance or the absorbance is determined. This can be done at a predetermined wavelength (for example the wavelength where the intensity is highest). Instead of measuring a change in peak in the spectrum, one could also determine a change in the integrated surface under the peak or measuring the shift in the spectrum. The measured transmittance or absorbance gives an indication of the presence of an analyte in the sample. E.g. when an analyte is present, the absorbance can increase or can decrease, the spectrum can shift, depending on the specific layers, on the interaction of the different layers, and on the analyte.

The transmittance or absorbance can be measured by a conventional absorbtiometer (also called calorimeter) or spectrometer. The measurement of absorbance or transmittance is advantageous compared to fluorescence measurements since no labelling of the molecule is required. Consequently, the experimental procedure as described in this invention is simplified. The use of a conventional light source such as a LED and the use of conventional absorptiometer result in method with a low manufacturing cost price.

The invention can be performed in a solution e.g. water based, such that a flow system can be used. Otherwise, the experiments can be performed as “dry measurements”.

Particularly in case of nano-particles, the absorption spectrum of metal nano particles is determined by both bulk interband absorption and particle plasmon resonances. The latter are collective oscillations of the conduction electrons on the surface of the small particle. The resonance frequency of a particle plasmon is determined mainly by the dielectric functions of the metal and the surrounding medium, respectively, and by the particle shape, i.e. the ratio of the principle axes. Resonances lead to narrow spectrally selective absorption and an enhancement of the local light field confined on and close to the surface of the metal particle. The surrounding medium influences the plasmon frequency and the amplitude of the absorption. The second mechanism playing a role in light absorption by small noble metal particles is photon interband absorption. It involves the promotion of an electron from the occupied d-level state in the noble metal to an empty state above the Fermi level. The absorption is strongly determined by the joint density of d and s states of the conduction electrons. Strong absorption indicates a “parallel” energy dispersion function. The different peaks n the spectrum can be assigned to different interband absorption peaks. Due to the large skin-depth of a few micron, the nanoparticles absorb light in the whole bulk area of the particle.

By increasing the dielectric constant near the nano particle surface an increase of the density for the electromagnetic field at the particles position enlarges the transition probability and as such the absorption for bulk transitions. This effect is only visible when the particle is smaller than the wavelength of the impinging light because such an object has too small a lateral extent to support any purely internal optical modes. The electric field operator internal to such a sphere is determined by the extended modes hence the dielectric constant of the surroundings.

As the dielectric constant increases, an increased absorption is expected as experimentally verified. This may be different for particle plasmons because the dielectric constant of the surroundings also has a strong influence on the wavenumber and strength of the collective and evanescent mode of excitations. By coating the particles with a different material both a shift in frequency and absorption probability is seen.

Compared to SPR measurements, the method as described in this application can have the several advantages. For example, the method can be more simple in set-up and can be made at a manufacturing low cost price. Moreover, this invention allows the different set-ups and can easily be integrated in different biological tools. In another advantage, for the measurements, a normal UV-vis spectrometer can be used. In yet another advantage, the intensity of the incoming light doesn't have to be focussed. A laser as incoming light is possible but is not necessary in this method.

FIG. 2 is a schematic representation of the method as described in conventional ELISA experiments. The method as described in the present invention has several advantages compared conventional ELISA experiment, wherein an antibody (21) is immobilized on a microtiterplate (23). This antibody cannot be detected by conventional UV-Vis measurements. The detection limit of UV-Vis measurements is not adequate to detect a thin layer or monolayer of proteins. In a next step the analyte or antigen (24) is recognized by the antibody. Also this event is not visible by UV-Vis measurements. Therefore a secondary antibody (25) with label (for example horseperoxidase) is used to couple to the other side of the antigen. Also this event is not visible. In a next step a substance is added which is converted by the label (HRP) to a colour in solution (26) which is or can give a quantitative estimation for the amount of antigen in the sample. This sequence of steps results in dilution curves, optimisations, calculations, time and money.

In the method as described in our invention, a thin layer of gold or nanoparticles are deposited on the bottom of an microtiterplate. The tin gold layer could be considered as being gold nano-particles. Gold particles are deposited on the bottom of the micotitreplate. In next step the antibody is coupled to the gold layer. The absorbance of the thin gold or the gold nanoparticles will be measured. This results in an absorption spectrum. In a next step the antibody is subjected to an external medium containing an antigen to be detected. The antigen will interact with the antibody resulting in a change in the absorption spectrum. The change can be an increase in intensity or a shift of the spectrum to lower/higher wavelengths Consequently, the method as described in the present invention makes the assay faster, simpler, cheaper and more reliable.

In one preferred embodiment, Ultrathin gold films were prepared via evaporation or via gold plating on a mercaptosilanized glass or quartz. The glass or quartz substrates were cleaned by dissolving them in 2 M NaOH for 2 hours followed by a 7 minutes treatment with a 1/1/5 mixture of respectively H₂O₂ (30%), NH₄0H (25%) and ultrapure H₂0 at 80 to 90° C. in order to achieve a fleshly prepared and uniform oxide layer.

3-Mercaptopropylmethyltrimethoxysilane was dissolved in a 95:5 (v/v) solvent mixture at 2%. The Self-Assembled mercaptosilane adhesion layers were formed by immersing of the substrates in this solution for up to 72 h. Following immersion, the substrates were removed from the solution and rinsed with methanol, blow-dried with N₂ and heated for 10 min at 110° C. The coated substrated were stored in N₂ until gold evaporation or platting.

For the preparation of the gold films, two techniques can be used: (i) the gold evaporation was performed at a speed <5 Å/sec with an Alcatel scm601. The final thickness on the mercaptosilanized substrates varied between 2 and 15 nm (average thickness), (ii) the electroless gold plating was performed as described in Jin et al. (Jin, Ye; Kang, X.; Song, Y.; Zhang, B,; Cheng, (G.; Dong, S, Anal. Chem. 2001, 73 (13), 2843). The mercaptosilanized substrates were overnight immersed in the colloidal gold solution mentioned above. The substrates having a monolayer of nanosized gold particles were consequently immersed in an aqueous 0.4 mM hydroxylamine hydrochloride and 0.1% HAuCl₄.3H₂0. All glassware was cleaned with 2 M NaOH for 2 hours. The substrates changed color from pink to purple to blue depending on the plating time therefore film thickness, After plating, the substrates were rinsed thoroughly with water, dried under a nitrogen stream, and were ready for measurements.

Self-Assembled Monolayers (SAMs) of 16-mercapto-1-texadecanoic acid (16-MHA), 1-octadecanethiol (HS-Cl8) and 1-dodecanethiol (HS-Cl2) were realized by immersing the clean ultrathin gold substrates in a 1 nM thiol/ethanol solution for various times. The slides were consequently rinsed successively with ethanol and dried under a stream of nitrogen.

UV/VIS spectroscopic studies were carried out using a Shimatzu UV-1601PC with a slit width of 2 nm and data interval of 0.5 nm. FIG. 3 is a schematic representation of slides and the quartz cells as described in the preferred embodiment of the present invention. The ultrathin gold-coated substrates were measured in air by placing the slides (31) perpendicular to the light beam. Characterization is solution was performed in the quartz cells (32), as shown in FIG. 3.

AFM surface images were acquired in tapping mode under ambient conditions (PicoSPM, Molecular Imaging, USA). Si cantilevers having a spring constant between 1.2 and 5.5 N/m were used at resonance frequencies between 60 and 90 kHz.

In a first experiment Human Serum Albumin was directly adsorbed on the thin gold film. FIG. 4(a) is an absorbance spectra of Human Serum Albumin directly adsorbed on the thin gold film. These measurements on evaporated thin gold on quartz were taken in ambient. FIG. 4(b) is a difference spectra Human Serum Albumin directly adsorbed on the thin gold film and a thin gold on quartz. These spectra are background-corrected with the background being the absorbance spectra of the thin gold film. The deposition of HSA on 4 nm of evaporated gold was performed by a drop of 1.244 mg/mL in PB for 120 min followed by thoroughly rinsing with water and drying under a stream of N₂. The next step was the deposition of a drop of anti-HSA 250 μg/mL in PB for 180 min with the same rinsing and drying procedure. The absorbance changes and shifts and after each biosensing step. The increase in peak is a measure for the concentration of anti-HSA.

Self-Assembeld Monolayers of thiols were used to induce the adsorption or to covalently attach the bioreceptor molecules to the ultrathin gold. In a next experiment we used quartz with 4 nm of evaporated gold. The UW measurements were performed in air. The thin gold layer was immersed for 90 min in a 10 mM 1-dodecanethiol—ethanol solution. Sequentially the adsorption of HSA in function of time was followed. Different concentrations and different times were used. The adsorption was performed from a drop of the different concentrations of HSA in HBS. FIG. 5(a) is an absorbance spectra of self-Assembeld Monolayers of thiols on gold followed by adsorption of HSA at different concentrations and different times. FIG. 5(b) is a difference spectra of the spectra of FIG. 5(a), The absorbance peak shift is shown in FIG. 5a, and the shift is more pronounced in the difference spectra in FIG. 5b. The dependence on the concentration is also clearly shown via the increase in the absorbance after introducing higher concentrations of HSA.

FIG. 6 is a difference spectra used in immunosensing applications. Immunosensing experiments were performed on a thin layer of platted gold (8 min of platting) and show clearly the potential of this sensing method for real biosensor application A Self-Assembled Monolayer of 16-MHA was formed on the thin gold film by a deposition of 25 minutes. The achieved carboxylic terminated SAM was activated via the EDC-NHS method with a mixture of 0.2 M/0.2 M EDC/NHS for 10 min. Consequently the amino groups of the lysine amino acids of anti-HSA (500 μg/mL in 10 mM acetate buffer pH=5) were covalently coupled to the activated SAM surface. The not-reacted activated groups were blocked by rinsing for 7 min with 1 M ethanolamine and the not covalently bonded antibodies were removed by 2 min rinsing with 10 mM glycine, HCl buffer pH=2.2. In this way a monolayer of anti-HSA on the surface can be observed. Again an enhancement around 270 nm is visible and a peak shift at 600 nm. This change in the spectra can be used to determine the concentration of anti-HSA.

In view of the wide variety of embodiments to which the principles of the present invention can be applied, it should be understood that the illustrated embodiments are exemplary only, and should not be taken as limiting the scope of the present invention Therefore, all embodiments that come within the scope and spirit of the following claims and equivalents thereto are claimed as the invention.

What is claimed is:

1. A method for detecting the presence of an analyte within a sample comprising:
providing a substrate, wherein the substrate comprises a conductive region and a recognition layer, the conductive region having a first surface and a second surface, the first surface being operatively coupled with the recognition layer, wherein the conductive region comprises at least one type of particle and the recognition layer is adapted to bind the analyte, and wherein the conductive region and recognition layer are selected and operatively coupled so as to result in a change in the resonance frequency of particle plasmons upon binding of the analyte to the recognition layer;
contacting the substrate with the sample to bind to the recognition layer at least a portion of the analyte that may be present in the sample;
directing radiation through the substrate wherein the principal wavelength of the impinging radiation is greater than the diameter of the at least one type of particle;
measuring from radiation transmitted through the sample at least part of the spectrum of the radiation that is absorbed by or transmitted through the substrate;
comparing the at least part of the spectrum to a reference spectrum, whereby a difference between the two spectra indicates binding of the analyte to the recognition layer and the presence of the analyte in the sample

2. The method as recited in claim 1, wherein the at least one type of particle has a diameter of less than 300 nm.

3. The method as recited in claim 1, wherein the binding of the analyte to the recognition layer results in a change in the dielectric constant of the recognition layer.

4. The method as recited in claim 1, wherein the substrate further comprises a support layer and the second surface of the conductive region is operatively coupled to the support layer.

5. The method as recited in claim 4, wherein the support layer is optically transparent to the radiation.

6. The method as recited in claim 4, wherein the support layer is optically semi-transparent to the radiation.

7. The method as recited in claim 1, wherein the conductive region comprises a metal.

8. The method as recited in claim 7, wherein the metal comprises at least one of gold, silver and copper.

9. The method as recited in claim 1, wherein the recognition layer comprises a linker layer and a recognition molecule

10. The method as recited in claim 1, wherein the recognition layer comprises a self-assembling monolayer.

11. The method as recited in claim 1, wherein the substrate has multiple conductive regions, the conductive regions being arranged in an array.

12. The method as recited in claim 1, wherein the substrate is a microtitre plate.

13. The method as recited in claim 1, wherein the reference spectrum is obtained by:
providing a second substrate;
subjecting the second substrate to a reference sample;
directing radiation through the second substrate;
measuring the intensity of the radiation absorbed or transmitted by or through the second substrate; and
comparing the intensity of the radiation absorbed or transmitted by the second substrate with the intensity of the radiation absorbed or transmitted by the first substrate in order to determine the presence of the analyte on the first substrate.

14. The method of claim 1, wherein the intensity of the radiation absorbed or transmitted by the substrate is determined as a function of a wavelength of the radiation.

15. The method according to claim I wherein the recognition layer is adapted to selectively bind the analyte.

16. The method according to claim 15, wherein the recognition layer comprises recognition molecules comprising one part of a specific binding pair selected from anti-gen/antibody, enzyme/substrate, metal/chelator, bacteria/receptor, virus/receptor, hormone/receptor, oligonucleotide/RNA, DNA/RNA, RNA/RNA, and oligonucleotide/DNA binding pairs.

17. The method of claim 1 wherein the reference spectrum is a spectrum of the substrate without adsorbed analyte.

18. The method of claim 1 further comprising determining the concentration of the analyte from the difference in the spectra.

19. The method according to claim 13, wherein the substrate of the reference spectrum does not contain any analyte.
